(19) United States
(12) Patent Application Publication
(10) Pub. No.: US 2007/0010580 A1
(43) Pub. Date: Jan. 11, 2007

(54) Formulation for chemical peeling

(76) Inventor: Gianfranco De Paoli Ambrosi, San Felice Del Benaco (IT)

Correspondence Address: SHOEMAKER AND MATTARE, LTD, 10 POST OFFICE ROAD - SUITE 110, SILVER SPRING, MD, 20910, US

(21) Appl. No.: 10/557,923

(22) PCT Filed: May 30, 2003
(86) PCT No.: PCT/IT03/00339

§ 371 Date: Mar. 10, 2006

(51) Int. Cl. A61K 31/225; A61K 8/37

(52) U.S. Cl. 514/547

(57) ABSTRACT

The present invention refers to a new formulation able to increase the efficacy and the tolerability of preparations based on the use of compounds used to carry out chemical peeling. More particularly, the present invention refers to a composition for chemical peeling, comprising at least one keratolytic agent and dimethyl isosorbide. The composition may additionally comprise dimethyl sulphone.

### FIELD OF THE INVENTION

The present invention relates to a new formulation able to increase the efficacy and the tolerability of preparations based on the use of compounds used to carry out chemical peeling.

### STATE OF THE ART

A very superficial peeling accelerates the natural exfoliation of the corneous layer, whilst a peeling which acts at a much deeper level causes necrosis and inflammation of the epidermis, the papillary dermis or of the reticular dermis.

Chemical peeling produces apparent changes in the skin through three mechanisms of action:

1. The stimulation of cellular turnover through the removal of the dead cells from the corneous layer.

2. The elimination of damaged and degenerated epidermal cells, which will be replaced by normal epidermal cells. This result will be particularly evident in the treatment of actinic keratosis and anomalous pigmentations.

3. The introduction of an inflammatory reaction and the activation of the mediators of inflammation (a mechanism still poorly understood) which activates the production of new collagen fibres and glycosaminoglycans (revitalising mechanisms of the dermis).

Since the peeling agents which operate at deep epidermal levels also carry the risk of complications and undesired outcome, it is indispensable to carry out treatments and therapies which achieve excellent results with the least possible risk.

Chemical peeling is particularly recommended in the following cases:

a) Keratosis and cutaneous ageing

b) Dischromia

c) Post—acne scarring

d) Common acne and rosacea

e) Radiodermatitis

f) Stretch marks

g) Seborrheic dermatitis

The various types of chemical peeling can be classified thus:
Very superficial peeling: this type of peeling only removes the superficial corneous layer;
Superficial peeling: this type of peeling causes necrosis of a part or in all of the epidermal layer reaching the basal layer of the epidermis;
Average depth peeling: this type of peeling causes necrosis of the epidermis and part of the papillary dermis;
Deep peeling: this type of peeling causes necrosis of the epidermis, the papillary dermis and can extend to the reticular dermis.

For chemical peeling the following chemical substances are generally used:

1. Retinoic acid

2. 5-Fluorouracile (5-Fu)

3. Jessner's Solution

4. Resorcine

5. Salicylic acid

6. Trichloroacetic acid

7. Alpha-hydroxy-acids

8. Alpha-keto-acids (such as for example pyruvic acid)

9. Phenol

The depth of peeling depends on numerous factors, such as: i) the type of substance used; ii) the concentration of the substance used, iii) the number of steps with the chosen substance on the same part of the skin, iv) the application technique, v) the preparation of the skin in the pre-treatment phase, vi) the type of cutaneous treatment in the period preceding the peeling, vii) the patients skin type, viii) the area of the cuteous treated, and ix) the exposure time to the selected chemical agent on the skin.

Considering all these variables, it is easy to understand that any classification relating to the various types of peeling has problems, since with the same substance we can obtain a superficial result on one type of skin, whilst on another type of skin we can obtain a medium or deep peeling. It is therefore not rare to cause even considerable damage to the skin, damage which, due to the variability delineated above, is frequently difficult to foresee.

The problem which lies at the heart of the present invention is therefore that of making available a formulation which allows on the one hand the attainment of an efficacious chemical peeling and which on the other hand minimises the risks of damage to the skin of the treated subject.

Such a problem is solved by a formulation for chemical peeling as delineated in the attached claims.

### DESCRIPTION OF THE INVENTION

It has been surprisingly found that the combination between the keratolytic agent and dimethyl isosorbide allows the attainment of an improvement in the absorption kinetics of the compound with keratolytic action; that leads to the use of lower quantities of keratolytic agent than those normally used despite achieving the same or more efficacious and efficient keratolytic action.

The use of lower quantities of keratolytic agent has as a consequence a drastic reduction of the recognisable side effects in the damage to the epidermis and to the dermis.

The present invention refers more specifically to a formulation for chemical peeling comprising a keratolytic agent in combination with dimethyl isosorbide. The dimethyl isosorbide will be present in such a quantity as to obtain an increase in the absorption kinetics of said keratolytic agent, if compared with the use of the keratolytic agent by itself. The increase in absorption kinetics is evaluated as shown below, i.e. by determining the quantity of keratolytic agent able to permeate through an SCE membrane by HPLC.

Preferred keratolytic compounds are selected from the chemical group of saturated and unsaturated monocarboxylic acids, saturated and unsaturated bicarboxylic acids, tricarboxylic acids, alpha hydroxyacids and beta hydroxyacids of monocarboxylic acids, alpha hydroxyacids and beta hydroxyacids of bicarboxylic acids, alpha hydroxyacids and beta hydroxyacids of tricarboxylic acids, ketoacids, alpha ketoacids, beta ketoacids, of the polycarboxylic acids, of the polyhydroxy monocarboxylic acids, of the polyhydroxy bicarboxylic acids, of the polyhydroxy tricarboxylic acids. Particularly preferred keratolytic agents are selected from the group comprising glycolic acid, tartaric acid, salicylic acid, citric acid, lactic acid, pyruvic acid, gluconic acid, glucuronic acid, malic acid, oxalic acid, malonic acid, succinic acid, acetic acid, phenol, resorcine, retinoic acid, adapalene, trichloroacetic acid, 5-fluoro uracil, azelaic acid. Keratolytic agents comprised within the scope of the present invention are also the salts, esters, possible cis or trans forms, racemic mixtures and/or the relative dextrorotatory or levorotatory forms of the above listed compounds. Such substances can be used singularly or in associations with each other.

According to a particularly preferred embodiment of the present invention the pharmaceutical and/or cosmetic composition of dimethyl isosorbide with one or more keratolytic agents comprises additionally dimethyl sulphone.

The dimethyl sulphone combined with keratolytic agents is capable of reducing the erythema induced by the agents themselves. According to this embodiment, the reduction of inflammation, irritation and erythema is obtained through the combination of the activity of the dimethyl sulphone with the fact that the quantity of keratolytic agent used to obtain the “peeling” effect is reduced thanks to the action of the dimethyl isosorbide. This latter component, as mentioned previously, increases the kinetics of percutaneous absorption of the keratolytic agent, rendering it more available for the action intended.

A third particularly preferred embodiment is that in which the keratolytic agent and/or the mixed keratolytic agents, combined with dimethyl isosorbide and dimethyl sulphone, are associated with the ester of an acid with keratolytic activity.

When used in association, the dimethyl isosorbide and the keratolytic agent and/or a mixture of keratolytic agents, can each be contained in the composition in a quantity by weight of from 1 to 99%, preferably each in a quantity comprised of between 5 and 40%. More preferably, dimethyl isosorbide and the keratolytic agents will be present in the composition in weight ratios comprised of between 1:4 and 4:1.

When used in association, the dimethyl isosorbide and the keratolytic agent and/or a mixture of keratolytic agents, associated with dimethyl sulphone can be contained in the compositions in a quantity by weight of from 1 to 99% each, preferably in a quantity comprised of between 5 and 70%. More preferably, dimethyl isosorbide and the keratolytic agent will be present in the composition in a ratio comprised of between 1:4 and 4:1. The dimethyl sulphone will preferably be present in a quantity comprised of between 2% and 70% by weight, more preferably between 10% and 65%, with respect to the keratolytic agent.

In the compositions of the invention, both based on keratolytic agent/dimethyl isosorbide and keratolytic agent/isosorbide/dimethyl sulphone, the weight balance up to 100% will be attained by the addition of solvents—such as water (in particular demineralised water), alcohols (such as ethyl alcohol) or glycols (for example, ethylene glycol or propylene glycol)—and/or excipients such as emulsifiers, antioxidants, lipid excipients, sequestrants, preservatives. Such excipients, used in particular for the preparation of emulsions, gels, creams, ointments, etc., are widely known to the expert in the field and will therefore not be described in any further detail.

Experiments relating to the evaluation of the favourable effect on percutaneous absorption of the keratolytic agent in the presence of dimethyl isosorbide is reported in corroboration of the present invention.

The aim of these experiments has been that of evaluating the in vitro percutaneous absorption across isolated human skin, of glycolic acid comprised in a formulation in which the keratolytic agent has been dissolved in water and propylene glycol (solution GC1) and another in which the keratolytic agent has been vehicularised using dimethyl isosorbide (solution GC2).

The experiment has been carried out using a system of Franz cells with a corneous-epidermis membrane layer (SCE membrane), the experimental protocol of which has already been widely described in the literature.

Preparation of the SCE Membranes

The preparation of the corneous-epidermis membrane layer (SCE) has been carried out using a technique already described in the literature, using samples of human skin originating from subjects, of ages comprised of between 32 and 45 years, subjected to reductive plastic surgery.

In these skin samples, following separation from the subcutaneous adipose layer and immersion in distilled water at a temperature of 60° C. for a few minutes, the dermis has been separated to obtain the SCE membranes used in this study. The removal of the dermis is made necessary because, in the in vitro evaluation of the percutaneous absorption of lipophilic substances, this tissue can be a “dummy” and additional barrier with respect to the in vivo cutaneous permeation process. The SCE membranes, thus prepared, have been dried and then placed in an appropriate desiccator. These membranes have then been conserved in sheets of aluminium at a temperature of around 4° C. and rehydrated at the time of use, by immersion in distilled water, one hour prior to the start of the permeation experiments. Prior to proceeding to the cutaneous permeation experiments and with the aim of evaluating the integrity of the SCE membranes used, the coefficient of permeability (Kp) of tritiated water has been determined for each sample of SCE membranes, the value of which is a sufficiently indicative parameter of the integrity of said membranes.

Cutaneous Permeation Experiments

For the evaluation of the degree of in vitro percutaneous absorption of glycolic acid from the formulations GC1 and GC2, batteries of six Franz cells (LGA, Berkeley, Calif.) have been used. Each Franz cell was constituted by a “donor” and a “receptor” between which has been placed the SCE membrane with the corneous layer facing the “donor”. The volume of the “receptor” of the cell was 4.7 ml whilst the surface area of the membrane in the “donor” (and therefore the potential cutaneous surface in contact with the product) was 0.75 cm².

The “receptor” compartment, stirred and thermostated at a temperature of 35-36° C., has been fed with an aqueous saline solution of 0.9% (w/v) NaCl. For the permeation experiment, 200 mg/cm² of each formulation GC1 and GC2, containing the glycolic acid, have been initially deposited onto each SCE membrane. The monitoring of the permeation process has then been carried out by determining, by a suitable HPLC method, the quantity of glycolic acid that has permeated through the corneous-epidermis (SCE) membrane layer over the 24 hour period following the application of the product into the “donor”. In order to carry out this experiment samples of SCE membranes originating from six different subjects (n=6) have been used, whilst each single permeation experiment has been performed in duplicate. The results have been expressed as the quantity of permeated glycolic acid, per cm² of skin, in 24 hours.

HPLC Determinations

The determinations of the quantities of glycolic acid, present in the receiver phase of the Franz cell 24 hours after the application of the formulations GC1 and GC2 have been carried out using an appropriate HPLC method reported in the literature.

Results

The results obtained (see Tab. 1) in the studies of the cutaneous permeation of glycolic acid from the formulations GC1 and GC2, demonstrate that the GC2 formulation is able to double (p<0.01) the quantity of glycolic acid permeated across the SCE membranes with respect to the GC1 formulation.

TABLE 1
The quantity of glycolic acid (expressed in μg/cm²) permeated from the
formulations GC1 and GC2 across the human skin (SCE) membranes
originating from six different subjects (A-F) in 24 hours.

| Subject | CG1 | CG2 |
|---|---|---|
| A | 65.7 | 143.8 |
| B | 82.7 | 169.2 |
| C | 39.4 | 95.3 |
| D | 75.1 | 95.2 |
| E | 112.5 | 197.3 |
| F | 76.1 | 235.4 |
| Mean | 75.2 | 156.0 |
| ± | 23.7 | 56.1 |

In the following are herein reported some examples of formulations according to the present invention.

Examples of Formulations

Preparation 1

| N° | Description | % (w/w) a |
|---|---|---|
| 01 | Dimethyl isosorbide | 50.00 |
| 02 | Pyruvic acid | 50.00 |

Preparation 2

| N° | Description | % (w/w) a |
|---|---|---|
| 01 | Dimethyl isosorbide | 40.00 |
| 02 | Pyruvic acid | 50.00 |
| 03 | Dimethyl sulphone | 10.00 |

Method of preparation: dissolve 03 in 01, to the solution obtained, mix in 02

Preparation 3

| N° | Description | % (w/w) a |
|---|---|---|
| 01 | Dimethyl isosorbide | 50.00 |
| 02 | Acetic acid | 50.00 |

Method of preparation: mix 01 and 02

Preparation 4

| N° | Description | % (w/w) a |
|---|---|---|
| 01 | Dimethyl isosorbide | 40.00 |
| 02 | Acetic acid | 50.00 |
| 03 | Dimethyl sulphone | 10.00 |

Method of preparation: dissolve 03 in 01, to the solution obtained, mix in 02

Preparation 5

| N° | Description | % (w/w) a |
|---|---|---|
| 01 | Dimethyl isosorbide | 75.00 |
| 02 | Trichloroacetic acid | 25.00 |

Method of preparation: mix 02 in 01

Preparation 6

| N° | Description | % (w/w) a |
|---|---|---|
| 01 | Dimethyl isosorbide | 75.00 |
| 02 | Trichloroacetic acid | 15.00 |
| 03 | Dimethyl sulphone | 10.00 |

Method of preparation: dissolve 03 in 01, to the solution obtained, mix in 02

Preparation 7

| N° | Description | % (w/w) a |
|---|---|---|
| 01 | Dimethyl isosorbide | 75.00 |
| 02 | Salicylic acid | 25.00 |

Method of preparation: mix 02 in 01

Preparation 8

| N° | Description | % (w/w) a |
|---|---|---|
| 01 | Dimethyl isosorbide | 75.00 |
| 02 | Salicylic acid | 15.00 |
| 03 | Dimethyl sulphone | 10.00 |

Method of preparation: dissolve 03 in 01, to the solution obtained, mix in 02

Preparation 9

| N° | Description | % (w/w) a |
|---|---|---|
| 01 | Dimethyl isosorbide | 20.00 |
| 02 | Tartaric acid | 30.00 |
| 03 | Propylene glycol | 15.00 |
| 04 | Water | 35.00 |

Method of preparation: dissolve 02 in 01, to the solution obtained, mix in 03+04

Preparation 10

| N° | Description | % (w/w) a |
|---|---|---|
| 01 | Dimethyl isosorbide | 30.00 |
| 02 | Glycolic acid | 60.00 |
| 03 | Water | 10.00 |

Method of preparation: dissolve 02 in 01. Mi the solution obtained with 03

Preparation 11

| N° | Description | % (w/w) a |
|---|---|---|
| 01 | Dimethyl isosorbide | 30.00 |
| 02 | Glycolic acid | 50.00 |
| 03 | dimethyl sulfone | 5.00 |
| 04 | Water | 15.00 |

Method of preparation: dissolve 02+03 in 01. Mix the solution obtained in 02

Preparation 12

| N° | Description | % (w/w) a |
|---|---|---|
| 01 | Dimethyl isosorbide | 50.00 |
| 02 | Resorcine | 10.00 |
| 03 | Salicylic acid | 20.00 |
| 04 | Ethyl alcohol | 20.00 |

Method of preparation: dissolve 02+03 in 04; to the solution obtained mix in 01

Preparation 13

| N° | Description | % (w/w) a |
|---|---|---|
| 01 | Dimethyl isosorbide | 60.00 |
| 02 | Resorcine | 10.00 |
| 03 | Salicylic acid | 20.00 |
| 05 | Ethyl alcohol | 10.00 |

Method of preparation: dissolve 02+03 in 05; to the solution obtained mix in 01

Preparation 14

| N° | Description | % (w/w) a |
|---|---|---|
| 01 | Dimethyl isosorbide | 50.00 |
| 02 | Retinoic acid | 1.00 |
| 03 | Salicylic acid | 20.00 |
| 04 | Dimethyl sulphone | 20.00 |
| 05 | Ethyl alcohol | 9.00 |

Method of preparation: mix 01+05; dissolve 02+03+04 in the solution obtained

Preparation 15

| N° | Description | % (w/w) a |
|---|---|---|
| 01 | Dimethyl isosorbide | 20.00 |
| 02 | Glycolic acid | 50.00 |
| 03 | Dimethyl sulphone | 10.00 |
| 04 | Ethyl lactate | 10.00 |
| 05 | Ethyl alcohol 95° | 5.00 |
| 06 | Propylene glycol | 1.00 |
| 07 | Demineralised water | 4.00 |

Method of preparation: dissolve 03+04 in 01: to the solution obtained mix in 02+05+06+07;

Preparation 16

| N° | Description | % (w/w) a |
|---|---|---|
| 01 | Dimethyl isosorbide | 30.00 |
| 02 | Lactic acid | 40.00 |
| 03 | Dimethyl sulphone | 10.00 |
| 04 | Ethyl lactate | 10.00 |
| 05 | Ethyl alcohol 95° | 5.00 |
| 06 | Propylene glycol | 1.00 |
| 07 | Demineralised water | 4.00 |

Method of preparation: dissolve 03+04 in 01: to the solution obtained mix in 02+05+06+07;

Preparation 17

| N° | Description | % (w/w) a |
|---|---|---|
| 01 | Dimethyl isosorbide | 50.00 |
| 02 | Trichloroacetic | 20.00 |
| | acid | |
| 03 | Dimethyl sulphone | 10.00 |
| 04 | Ethyl pyruvate | 10.00 |
| 05 | Ethyl alcohol 95° | 5.00 |
| 06 | Propylene glycol | 1.00 |
| 07 | Demineralised water | 4.00 |

Method of preparation: dissolve 03+04 in 01: to the solution obtained mix in 02+05+06+07;

Preparation 18

| N° | Description | % (w/w) a |
|---|---|---|
| 01 | Dimethyl isosorbide | 50.00 |
| 02 | Salicylic acid | 20.00 |
| 03 | Dimethyl sulphone | 10.00 |
| 04 | Ethyl pyruvate | 10.00 |
| 05 | Ethyl alcohol 95° | 5.00 |
| 06 | Propylene glycol | 1.00 |
| 07 | Demineralised water | 4.00 |

Method of preparation: dissolve 03+04 in 01: to the solution obtained mix in 02+05+06+07;

Preparation 19

| N° | Description | % (w/w) a |
|---|---|---|
| | PHASE A | |
| 01 | Steareth 2 | 3.00 |
| 02 | Steareth 21 | 2.00 |
| 03 | Ppg 15 stearyl ether | 10.00 |
| 04 | Tocopheryl acetate | 1.00 |
| 05 | Jojoba oil | 2.00 |
| 06 | Bht | 0.01 |
| 07 | Ascorbyl palmitate | 0.10 |
| 08 | Ethyl pyruvate | 5.00 |
| | PHASE B | |
| 08 | Propylene glycol | 2.00 |
| 09 | Pyruvic acid | 10.00 |
| 10 | Demineralised water | 10.00 |
| | PHASE C | |
| 11 | Dimethyl sulphone | 10.00 |
| 12 | Propylene glycol | 2.00 |
| 13 | Disodium EDTA | 0.07 |
| 14 | Glycerol | 5.00 |
| 15 | Phenoxyethanol | 1.00 |
| 16 | Methyl paraben | 0.10 |
| 17 | Ethyl paraben | 0.10 |
| 18 | Propyl paraben | 0.10 |
| 19 | Demineralised water qba | 100 |

Method of preparation: heat PHASE A) to 75° C.; heat PHASE C to +75° C.; combine PHASE A with PHASE C with stirring homogenising the solution; cool to +45° C.; then combine with PHASE B still with stirring and cool to 25° C.

Preparation 20

| N° | Description | % (w/w) a |
|---|---|---|
| | PHASE A | |
| 01 | Steareth 2 | 3.00 |
| 02 | Steareth 21 | 2.00 |
| 03 | Ppg 15 stearyl ether | 10.00 |
| 04 | Tocopheryl acetate | 1.00 |
| 05 | Jojoba oil | 2.00 |
| 06 | Bht | 0.01 |
| 07 | Ascorbyl palmitate | 0.10 |
| 08 | Ethyl lactate | 5.00 |
| | PHASE B | |
| 08 | Propylene glycol | 2.00 |
| 09 | Tartaric acid | 15.00 |
| 10 | Demineralised water | 10.00 |
| | PHASE C | |
| 11 | Dimethyl sulphone | 10.00 |
| 12 | Propylene glycol | 2.00 |
| 13 | Disodium EDTA | 0.07 |
| 14 | Glycerol | 5.00 |
| 15 | Phenoxyethanol | 1.00 |
| 16 | Methyl paraben | 0.10 |
| 17 | Ethyl paraben | 0.10 |
| 18 | Propyl paraben | 0.10 |
| 19 | Demineralised water qba | 100 |

Method of preparation: heat PHASE A) to 75° C.; heat PHASE C to +75° C.; combine PHASE A with PHASE C with stirring homogenising the solution; cool to +45° C.; then combine with PHASE B still with stirring and cool to 25° C.

Preparation 21

| N° | Description | % (w/w) a |
|---|---|---|
| | PHASE A | |
| 01 | Steareth 2 | 3.00 |
| 02 | Steareth 21 | 2.00 |
| 03 | Ppg 15 stearyl ether | 10.00 |
| 04 | Tocopheryl acetate | 1.00 |
| 05 | Jojoba oil | 2.00 |
| 06 | Bht | 0.01 |
| 07 | Ascorbyl palmitate | 0.10 |
| 08 | Zinc oxide oily solution | 20.00 |
| | 50% | |
| | PHASE B | |
| 08 | Propylene glycol | 2.00 |
| 09 | Lactic acid | 10.00 |
| 10 | Demineralised water | 10.00 |
| | PHASE C | |
| 11 | Dimethyl sulphone | 10.00 |
| 12 | Propylene glycol | 2.00 |
| 13 | Disodium EDTA | 0.07 |
| 14 | Glycerol | 5.00 |
| 15 | Phenoxyethanol | 1.00 |
| 16 | Methyl paraben | 0.10 |
| 17 | Ethyl paraben | 0.10 |
| 18 | Propyl paraben | 0.10 |
| 19 | Demineralised water qba | 100 |

Method of preparation: heat PHASE A) to 75° C.; heat PHASE C to +75° C.; combine PHASE A with PHASE C with stirring homogenising the solution; cool to +45° C.; then combine with PHASE B still with stirring and cool to 25° C.

In the present invention, the mixture of dimethyl isosorbide, associated with a keratolytic agent and/or a mixture of keratolytic agents, together with dimethyl sulphone, can be combined with esters of the keratolytic agents, preferably the ethyl esters. The use of the keratolytic agent esters, singularly and/or in association, is justified by the fact that, once absorbed into the cuteous, these are hydrolysed liberating the acid and alcohol. The acid form will therefore be able to continue the keratolytic action, in a less intense form but with a more protracted effect over time. Examples of the esters of the keratolytic agents are ethyl pyruvate, ethyl glycolate, triethyl citrate, ethyl resorcinate, the ethyl ester of retinoic acid, ethyl salicylate, methyl salicylate, ethyl malnate, ethyl acetate, ethyl tartrate.

A further subject of the present invention is a formulation for chemical peeling comprising of one or more keratolytic agents, preferably selected from the above described group, together with a keratolytic agent ester. The ester of the keratolytic agent will preferably be selected from the above listed group and can be the ester of the same keratolytic agent used in acid form or the ester of a different keratolytic agent. Such keratolytic agent esters will be present in the composition in a quantity preferably comprised of between 3% and 60% by weight, more preferably in a quantity comprised of between 15% and 50% by weight, with respect to the keratolytic agent (or to the mixture of keratolytic agents).

In place of the keratolytic agent esters, another derivative or pro-drug can however be used, in the same proportions indicated above, which is capable of liberating after administration under biological conditions, the keratolytic agent in the treatment site.

It is evident that the combination of one or more keratolytic agents and their derivative or pro-drug as defined above can also be applied to compositions in which the dimethyl isosorbide or the dimethyl sulphone are not present. One will in fact obtain, in any case, the desired effect of achieving a more prolonged over time and at the same time less acute chemical peeling, with the consequent reduction of the irritant phenomena caused by intense and acute treatment.

1-20. (canceled)

21. A composition for chemical peeling, comprising at least one keratolytic agent and dimethyl isosorbide.

22. The composition according to claim 21, wherein said keratolytic agent, alone or as a mixture of two or more keratolytic agents, and said dimethyl isosorbide are each contained in a quantity of between 1% and 99% by weight.

23. The composition according to claim 22, wherein said keratolytic agent, alone or as a mixture of two or more keratolytic agents, and said dimethyl isosorbide are each contained in a quantity comprised of between 5% and 40%.

24. The composition according to claim 21, wherein said keratolytic agent, alone or as a mixture of two or more keratolytic agents, and said dimethyl isosorbide are contained in a weight ratio comprised of between 1:4 and 4:1.

25. The composition according to claim 21, wherein said at least one keratolytic agent is selected from saturated and unsaturated monocarboxylic acids, saturated and unsaturated bicarboxylic acids, tricarboxylic acids, alpha hydroxyacids and beta hydroxyacids of monocarboxylic acids, alpha hydroxyacids and beta hydroxyacids of bicarboxylic acids, alpha hydroxyacids and beta hydroxyacids of tricarboxylic acids, ketoacids, alpha ketoacids, beta ketoacids, polycarboxylic acids, polyhydroxy monocarboxylic acids, polyhydroxy bicarboxylic acids, polyhydroxy tricarboxylic acids, salts, esters, possible cis or trans forms, racemic mixtures and/or relative dextrorotatory or levorotatory forms thereof.

26. The composition according to claim 25, wherein said at least one keratolytic agent is selected from glycolic acid, tartaric acid, salicylic acid, citric acid, lactic acid, pyruvic acid, gluconic acid, glucuronic acid, malic acid, oxalic acid, malonic acid, succinic acid, acetic acid, phenol, resorcine, retinoic acid, adapalene, trichloroacetic acid, 5-fluoro uracil, azelaic acid.

27. The composition according to claim 21, further comprising cosmetically or pharmaceutically acceptable solvents and/or cosmetically or pharmaceutically acceptable excipients.

28. The composition according to claim 27, wherein said solvents are selected from water, alcohols or glycols and said excipients are selected from emulsifiers, antioxidants, lipid excipients, sequestrants, preservatives.

29. The composition according to claim 21, said compositions further comprising dimethyl sulphone.

30. The composition according to claim 29, wherein said dimethyl sulphone is contained in a quantity comprised of between 2% and 70% by weight with respect to the keratolytic agent.

31. The composition according to claim 30, wherein said dimethyl sulphone is contained in a quantity comprised of between 10% and 65% by weight with respect to the keratolytic agent.

32. The composition according to claim 21, said composition further comprising a derivative or pro-drug of said at least one keratolytic agent which is able to liberate, following administration, the keratolytic agent under the biological conditions of the site for treatment.

33. The composition according to claim 32, wherein said derivative or pro-drug is an ester of said at least one keratolytic agent, or as an ester of the same keratolytic agent used in the compositions or as an ester of a different keratolytic agent.

34. The composition according to claim 33, said ester of said at least one keratolytic agent being selected from ethyl pyruvate, ethyl glycolate, triethyl citrate, ethyl resorcinate, the ethyl ester of retinoic acid, ethyl salicylate, methyl salicylate, ethyl malnate, ethyl acetate, ethyl tartrate.

35. The composition according to claim 32, wherein said derivative or pro-drug of said at least one keratolytic agent is contained in a quantity comprised of between 3% and 60% by weight with respect to the keratolytic agent.

36. The composition according to claim 32, wherein said derivative or pro-drug of said at least one keratolytic agent is contained in a quantity comprised of between 15% and 50% by weight with respect to the keratolytic agent.

37. A method for the chemical peeling of the skin, wherein the skin is treated with a composition comprising a keratolytic agent and dimethyl isosorbide, wherein said dimethyl isosorbide increases the absorption kinetics of said keratolytic agent.

38. A method for the chemical peeling of the skin, wherein the skin is treated with a composition comprising dimethyl sulphone as an anti-inflammatory, anti-irritant and antierythema agent.

39. A composition for chemical peeling, comprising at least one keratolytic agent and a derivative or pro-drug of a keratolytic agent which is able to liberate, following administration, the keratolytic agent under the biological conditions of the site for treatment.

40. The composition according to claim 39, said derivative or pro-drug being an ester of a keratolytic agent.
